(19) United States
(12) Patent Application Publication
(10) Pub. No.: US 2018/0146645 A1
(43) Pub. Date: May 31, 2018

(54) SYSTEM AND METHOD FOR MONITORING LIVESTOCK

(71) Applicant: CATTLE-WATCH LTD, REHOVOT (IL)

(72) Inventor: ILAN ARBEL, REHOVOT (IL)

(21) Appl. No.: 15/575,820

(22) PCT Filed: Apr. 12, 2016
(86) PCT No.: PCT/IL2016/050385

§ 371 Date: Nov. 21, 2017

Related U.S. Application Data
(60) Provisional application No. 62/276,281, filed on Jan. 8, 2016.
(60) Provisional application No. 62/166,028, filed on May 25, 2015.

(51) Int. Cl. A01K 29/00; G06Q 50/02; A01K 11/00

(52) U.S. Cl. CPC A01K 29/005; G06Q 50/02; A01K 11/008; A61B 2503/40; A01K 11/001

(57) ABSTRACT

A system and method for monitoring livestock, the system including a remote server storing data, the remote server including a processing unit for processing stored data and a non-volatile memory; at least one simple data collection device for mounting on an animal to be monitored, the data collection device including a sensor sensing physical parameters of the animal on which it is mounted, a simple data collection device processor with a non-volatile memory, and a transmitter for transmitting data collected by the sensor; at least one central data collection device including a mobile hub for mounting on an animal to be monitored, the central data collection device including: a central data collection device processor with a non-volatile memory; a receiver for receiving data transmitted by the simple data collection devices; a transceiver for communication with the remote server over a communication system; an energy source; the remote server being configured to analyze collected physical parameters and determine therefrom physical condition or behavior of the animal; and a network for two-way communication between the remote server and a remote electronic communication device and configured to provide real time information and warning alarms to the remote electronic communication device.

### FIELD OF THE INVENTION

The present invention relates to a system for monitoring livestock, in general, and, in particular, to a method and system for monitoring livestock and other assets at a remote location.

### BACKGROUND OF THE INVENTION

Ranchers currently lack cost effective means to monitor the physical condition of their cattle across great distances and large grazing areas. The result is low yield, meaning the number of calves born in a given year that survive the six to nine month nursing period. In addition, in the modern world, about 5% of cattle head is lost every year due to health issues. In some countries, herds roam freely and graze in very large areas, usually without communication network coverage, making it very difficult to keep track of the various members of the herd.

Monitoring herd health status has been of major interest to the beef industry. Some systems have been provided over the years, including devices which were mounted on all the animals in the herd, relying on the fact that some of the herd at some of the time will be in an area covered by a communication network. These systems usually require high maintenance, for example, in supplying an energy source, since each animal in the herd must be accessed for this maintenance. In addition, due to the complexity of accessing each animal, the devices mounted on these animals are usually as simple as possible, and can provide only very basic raw information.

There are also known drones for flying over distant areas and providing images from an airborne camera for tracking and monitoring livestock.

Accordingly, there is a long felt need for a system that permits remote monitoring of individual livestock in herds, and it would be very desirable if such a system could provide an indication of selected physical conditions of the livestock in real time from a remote location.

### SUMMARY OF THE INVENTION

The present invention relates to a remote monitoring system for monitoring selected physical conditions and behaviors of livestock across long distances and over large grazing areas in open pastures, as well as in fenced in areas. The system includes several types of electronic data collection devices, one of which is mounted on each animal. Most of the electronic data collection devices are in-herd network devices, which form a communication network between devices on livestock in that herd, only. Each device includes an identification number that identifies the individual animal as well as the herd to which it belongs. These devices can be relatively simple collars or ear tags, which include identification data and sensors to record various physical parameters of the animal, such as, motion, posture and speed, etc., of the animal, from which selected physical conditions and behaviors of the animal can be determined. A relatively small percentage of the electronic data collection devices, for example 5-7%, are mobile hub devices, which receive the information from the in-herd network devices. These mobile hub devices also include animal identification data and a sensor for monitoring the animal on which they are mounted. These mobile hub devices move randomly (i.e., not over a fixed or pre-determined route) while the animal wearing it roams. Each hub device is further provided with a transmitter to transmit the data in real time to a remote server, for example, via a satellite, cellular or GPRS network. The server, in turn, transmits the data to a user's personal computer (PC) and/or cellular phone. It will be appreciated that the in-herd network devices can be low-power devices, as they transmit only over short distances to the mobile hub devices. On the other hand, the mobile hub devices require an energy source to permit transmission of all the data from the in-herd network to a satellite or other network for further dissemination.

In addition, an early warning alarm or other notification, when illness or hostile events are determined from the physical parameters detected by the sensors, can be sent directly to the user's cellular phone or other communication device. In this case, the user, via the server, can send a drone to the relevant area to capture and transmit video images of the herd to the user's cellphone or PC. The drone's flight can be operated and controlled automatically, with no manual intervention required. The drone can include a thermal camera, as well as a visible spectrum camera, to operate at night as well as during the day.

Thus, there is provided, according to the present invention, a system for monitoring animals to be monitored including a remote server storing data, the remote server including a processing unit for processing stored data and a non-volatile memory; at least one simple data collection device for mounting on an animal to be monitored, the data collection device including a sensor sensing physical parameters of the animal on which it is mounted, a simple data collection device processor with a non-volatile memory, and a transmitter for transmitting data collected by the sensor; at least one central data collection device including a mobile hub for mounting on an animal to be monitored, the central data collection device including: a central data collection device processor with a non-volatile memory; a receiver for receiving data transmitted by the simple data collection devices; a transceiver for communication with the remote server over a communication system; an energy source; the remote server being configured to analyze collected physical parameters and determine therefrom physical condition or behavior of the animal; and a network for two-way communication between the remote server and a remote electronic communication device and configured to provide the analyzed data in real time and warning alarms to the remote electronic communication device.

There is further provided, according to the present invention, a method for monitoring animals to be monitored, the method including collecting, at pre-defined time intervals for pre-defined periods of time, data of physical parameters of an animal to be monitored sensed by a sensor in a simple data collection device mounted on the animal to be monitored; storing the collected data in the simple data collection device; transmitting stored collected data by the simple data collection device to a mobile hub device at pre-defined time intervals; receiving, in the mobile hub device, the data transmitted by the simple data collection device, transmitting, by the mobile hub device, the received data to a remote server; analyzing the transmitted data to determine physical condition and behavior in the remote server and storing the analyzed data; and permitting access to the stored data in the remote server by at least one remote electronic communication device, the remote server transmitting real time information and warning alarms to the remote electronic communication device.

### BRIEF DESCRIPTION OF THE DRAWINGS

The present invention will be further understood and appreciated from the following detailed description taken in conjunction with the drawings in which:

FIGS. 1a, 1b and 1c are schematic illustrations of exemplary embodiments of a livestock monitoring system constructed and operative in accordance with the present invention;

FIG. 2a is a block diagram illustration of an electronic data collection device, according to embodiments of the invention;

FIGS. 2b and 2c are schematic illustrations of electronic data collection devices according to embodiments of the invention, in use;

FIG. 3 an exemplary screen shot displaying data of a remote herd on the display of a personal lap-top computer; and

FIG. 4 is an exemplary screen shot displaying data of a remote herd on a cellular telephone including a warning alarm;

FIG. 5 is a schematic illustration of the use of the system of the present invention as a feedlot theft early warning system.

FIG. 6 is a schematic illustration of use of the system for counting heads of livestock;

FIG. 7 is a schematic illustration of a system for monitoring livestock and also measuring water level in a drinking hole, according to embodiments of the invention;

FIG. 8 is an illustration of a matrix useful in an algorithm for determining physical characteristics of an animal, according to embodiments of the invention;

FIG. 9a is a schematic illustration of sensor results indicating behaviors of an animal, according to embodiments of the invention; and

FIG. 9b is an exemplary illustration of sensor results indicating in heat behavior of an animal, according to embodiments of the invention.

### DETAILED DESCRIPTION OF THE INVENTION

The present invention relates to a remote monitoring system for monitoring one or more physical conditions or behaviors of livestock roaming over long distances and large grazing areas by sensing and analyzing selected physical parameters of each animal. This can be accomplished by sensors that collect data regarding physical parameters, such as motion of the animal or portions of its body, posture, speed, attitude of body parts, motion of the neck, angle of the back relative to the ground, etc. The system and method of operation permit a user to identify the physical condition or behavior, such as, standing, lying, grazing, mating, and so forth, of livestock by measuring periodically these physical parameters of the members of small and medium size herds of livestock (tens to tens of thousands of heads). For ease of description, the system and method will be described herein with relation to cattle, for which it is particularly suited. However, it will be appreciated that it is equally applicable to herds of other livestock (sheep, goats, horses, etc.), that roam free on remote pastures or require monitoring from a remote location. Thus, the illustrated exemplary embodiment of the system provides periodic, (such as once a day, every hour, etc.) real time information of one or more selected physical parameters of cows, bulls and calves, permitting determination of physical conditions or behaviors, for example, standing, walking, lying down, in heat, pregnancy, illness, bull efficiency, calf delivery and calf condition. In addition, the system permits tracking and tracing of the geographical location of each member of the herd during grazing, etc., and can provide an early warning of illness or of theft or other hostile activity, and save operational costs. By providing daily information about the physical condition of cows, bulls and calves, for example, as well as location and tracking information, yields can be increased up to 25%.

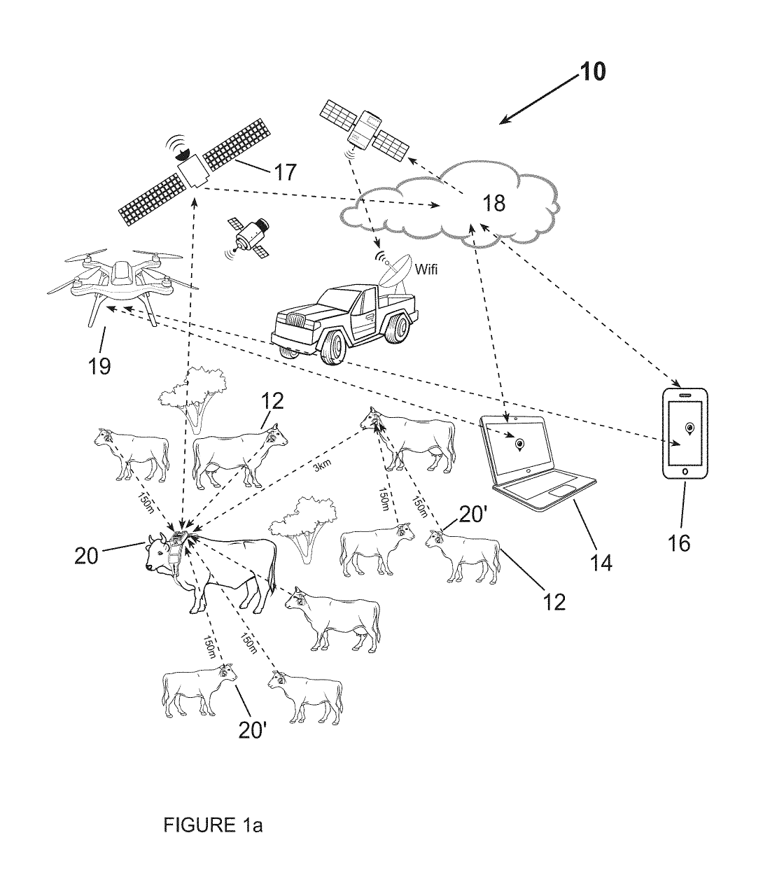

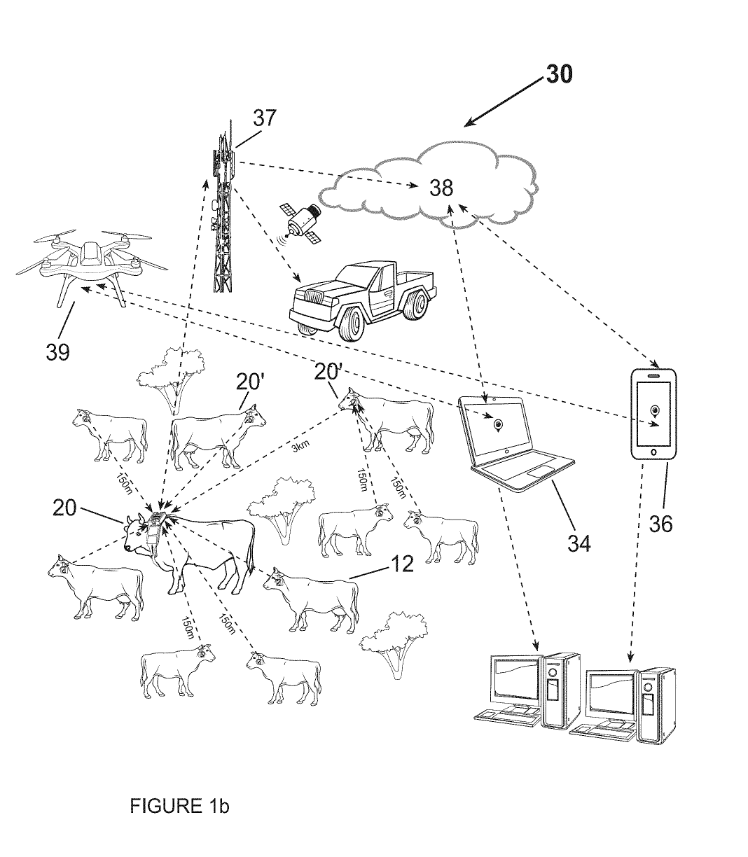

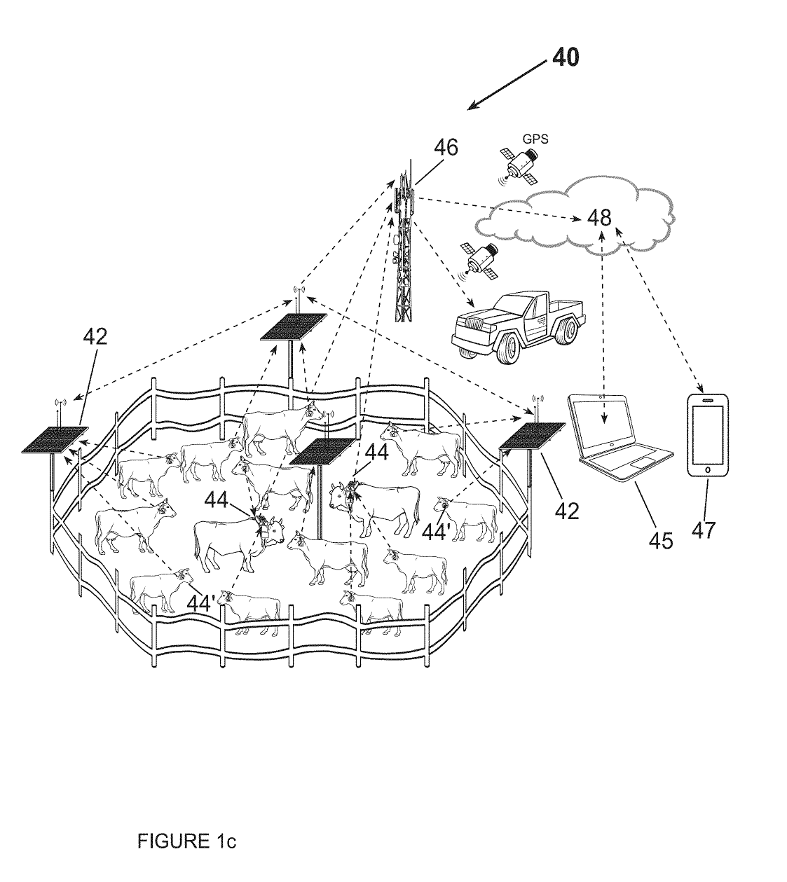

Referring now to FIG. 1a, there is shown a schematic illustration of a livestock monitoring system 10 constructed and operative according to exemplary embodiments of the invention. System 10 is satellite based and utilizes Internet cloud-based sensor technology that permits a user to track and monitor animals 12 from the comfort of home or office, using a personal computer 14 or a smart phone 16. An electronic data collection device 20, 20′ is mounted on each animal 12 to be monitored. Data collection device 20 is in two-way communication over a communication network, such as via a satellite 17, such as an Iridium satellite, with a remote server 18 in the Internet cloud, which, in turn, is in one- or two-way communication over the communication network with the user's PC 14 and/or smart phone 16. Satellite 17 permits transmission of the data to substantially any location on the globe. If Iridium satellites are employed, it will be appreciated that more than one satellite can be above the herd being monitored at one time. A drone 19 can be employed, as described in detail below, to fly over the herd and collect video images or other images to be transmitted to the server, e.g., for display on the user's PC 14 and/or smart phone 16.

FIG. 1b shows a schematic illustration of a livestock monitoring system 30 constructed and operative according to exemplary embodiments of the invention. System 30 utilizes a cellular or GPRS (General Packet Radio Service) based communication system, without a satellite, but also including a management system utilizing a PC 34 and/or a smart phone 36. Thus, system 30 also includes a plurality of electronic data collection devices 20, 20′ mounted on the animals 12 to be monitored. In this embodiment, data collection devices 20 are in two-way communication via a GPRS base station 37 and GPRS network with a remote server 38 in the Internet cloud, which, in turn, is in one- or two-way communication with the user's PC 34 and/or smart phone 36 over a communication network. In this embodiment, as well, a drone 39 can be operated by the server to fly over the herd and collect video images or other images to be transmitted for display on the user's PC 34 and/or smart phone 36.

FIG. 1c illustrates, schematically, a livestock monitoring system 40 constructed and operative according to alternative embodiments of the invention. System 40 utilizes a cellular or GPRS (General Packet Radio Service) based communication system together with a physical mesh of stationary terminals 42 having transceivers. System 40 includes a plurality of simple cellular electronic data collection devices 44′ mounted on the animals 12 to be monitored. The data collection devices 44′ are capable of transmitting only a relatively short distance. The data collected by their sensors is transmitted to one of a plurality of local terminals 42 disposed around an area where the livestock congregate or are fenced in. Local terminals 42 are arranged for communication with the cellular or GPRS base station 46, either directly or through a collecting terminal, and serve the same function as the mobile hub devices of FIG. 1a. Alternatively, or in addition, a few mobile hub data collection devices 44 can be provided to transmit collected data to the GPRS or cellular base station 46. The cellular or GPRS base station 46 communicates with a remote server 48 in the Internet cloud, which, in turn, is in one- or two-way communication with the user's PC 45 and/or smart phone 47.

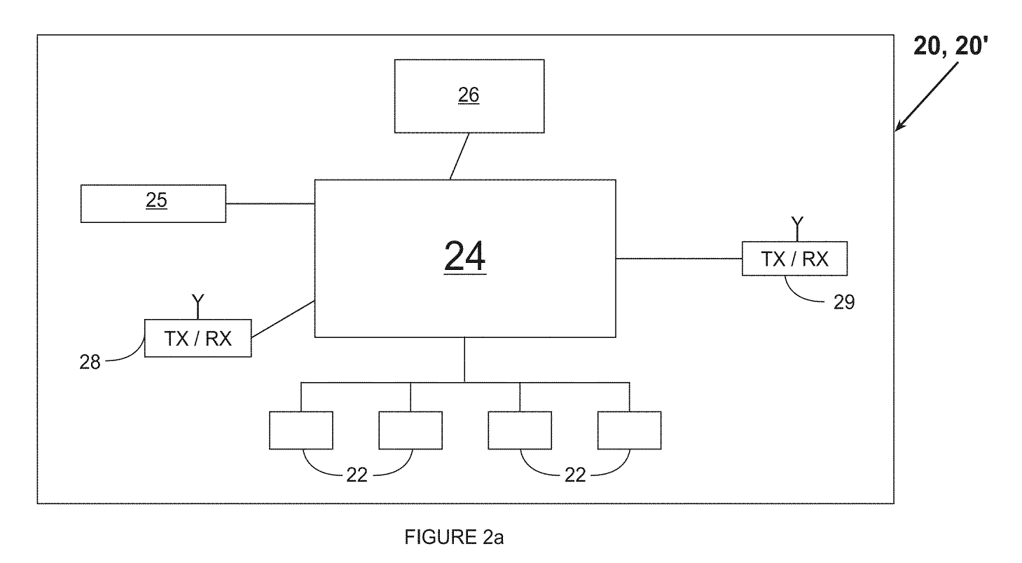

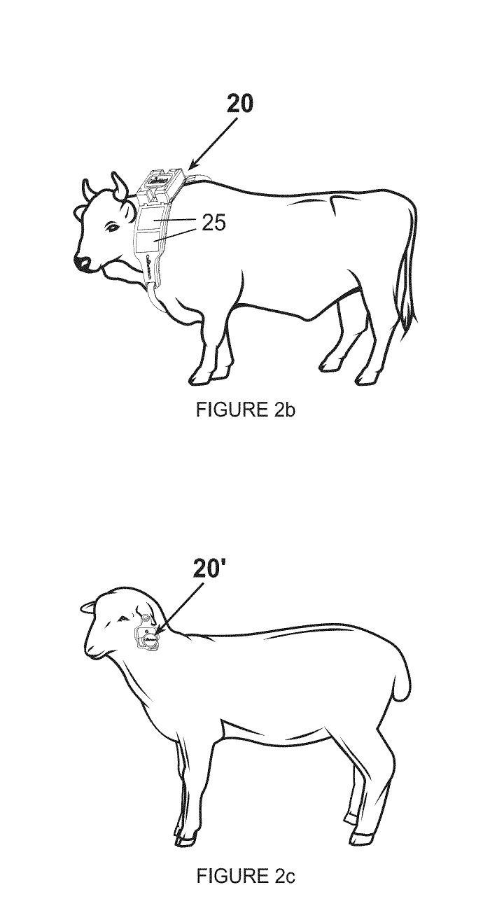

Referring now to FIG. 2a, there is shown a block diagram illustration of an electronic data collection device 20, 20′, according to embodiments of the invention, suitable for use in the networks of any one of FIGS. 1a, 1b and 1c. Data collection device 20 includes at least one, and preferably a plurality of sensors 22 for sensing physical parameters, such as motion, position, attitude or position of body parts, speed of movement, and/or other selected parameters of the animal on which is it mounted, and a processor 24 with a non-volatile memory 26 for receiving the data from the sensors 22 and storing them until transmitted to a data collection location. Thus, the sensor device may include, one or more of, for example, a 3-axis acceleration sensor, a 3-axis gyro sensor, inertial sensors, an optional GPS device, a motion sensor, an altimeter, etc., that record various details of position, posture and physical parameters of the livestock. Most of the simple data collection devices 20′ include a radio frequency transmitter or transceiver 28 (e.g., 600-900 meters), or a short range transmitter or transceiver, such as Bluetooth or Infrared, for one-way or two-way in-herd communication, to transmit collected data to nearby receivers in central data collection devices 20, thereby creating a wireless in-herd communication network. These sensor devices 20′ can be mounted in a non-solar power based ear tag or a collar to be worn by the animal. An energy source 25 is provided to power all the electronic components of the data collection device. The energy source 25 in a simple collection device 20′ can be, for example, a long-life battery, e.g., a non-rechargeable battery, such as a primary lithium battery, a solar charging panel, preferably of the sort capable of operating for a year or more, so that frequent replacement is not required.

A small percentage of the data collection devices are hub devices 20, mounted on an animal to be monitored, which collect data while the animal roams randomly over the grazing area, and will be referred to herein as mobile hub devices. Mobile hub devices 20 receive data from the various simple data collection devices 20′ in their vicinity. These hub devices 20 also include a long distance transceiver 29 for transmitting the collected data to a remote location, such as server 18, which may be in the Internet cloud. Preferably, the energy source 25 of the mobile hub devices 20 includes one or more photovoltaic panels, that can be built-in or mounted in the device, that harvest solar energy for storage in industrial-grade Li-ion rechargeable batteries. These batteries deliver the electric pulses needed to ensure satellite-, cellular- or GPRS-based real-time communications between the in-herd mesh network and the server or the user.

The data collection devices can be mounted, for example, in an ear tag or on a collar for tying to the neck of the animal, or can be mounted inside the animal or under its skin. One example of a central data collection device 20, here illustrated as a collar, in use, is illustrated in FIG. 2b. The collar can be tied, clipped or buckled in any way around the neck of the animal, as long as the housing of the components rests on the animal's spine and the solar panels 25 are exposed to the sun. A simple data collection device 20′ illustrated in FIG. 2c as an ear tag. Each data collection device includes an identification number or other means of uniquely identifying the animal on which the device is mounted. This identification number identifies both the individual animal and the herd to which it belongs.

The system permits tracking and tracing of the location of each member of the herd during grazing, etc., providing an early warning of illness or of theft or other hostile activity, and saving operational costs. This is accomplished by providing to the user (e.g., farmer, rancher) a daily report of activity of deterministic binary events of each bull, cow and calf. For purposes of the invention, these deterministic binary events include at least one of the following physical conditions and behaviors:

For a bull: walking; breaking a leg; jumping on a cow; grazing; drinking; lying down; standing; running; restlessness; panic (hostility).

For a cow wearing a hub collar or a simple collar: walking; grazing; breaking a leg; drinking; lying down; running; standing; feeding (nursing); panic (hostility); restlessness; in heat condition; pregnancy; deliver calf; abortion.

For a calf with a simple collar or an ear tag: walking; grazing; breaking a leg; nursing (drinking milk or getting food); drinking water; lying down; standing; restlessness; panic (hostility).

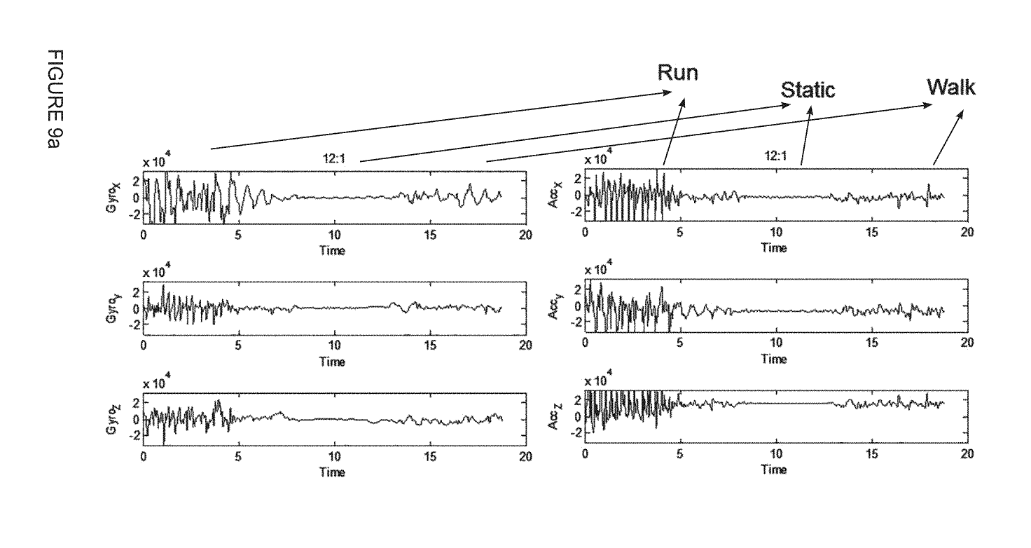

Detection of these events is accomplished by analyzing the data collected by the sensors in the electronic data collection devices, for example, a 3 axis accelerator and a 3 axis gyro. The sensed data is collected in the data collection devices and analyzed in the remote server by decoding the signals of the physical parameters and determining each event by a singular mathematical algorithm that identifies various events. Given continuous data from the accelerator and gyro on the animal's neck, it is possible to categorize various parameters of the animal. The data collection device attitude, which reflects the neck attitude, can be calculated by the following Direction Cosine Matrix (DCM):

$${R}_{I}^{B} ( φ , θ , ψ ) = ( \begin{matrix}c ( ψ ) c ( θ ) & c ( θ ) s ( ψ ) & - s ( θ ) \\ c ( ψ ) s ( φ ) s ( θ ) - c ( φ ) s ( ψ ) & c ( φ ) c ( ψ ) + s ( φ ) s ( ψ ) s ( θ ) & c ( θ ) s ( φ ) \\ s ( φ ) s ( ψ ) + c ( φ ) c ( ψ ) s ( θ ) & c ( φ ) s ( ψ ) s ( θ ) - c ( ψ ) s ( φ ) & c ( φ ) c ( θ )\end{matrix} )$$

where ϕ represents the roll, in the X axis, θ represents the pitch, in the Y axis, and ψ represents the yaw, in the Z axis. See, for example, FIG. 8, which illustrates the elements of this matrix for determining physical parameters of an animal, according to embodiments of the invention.

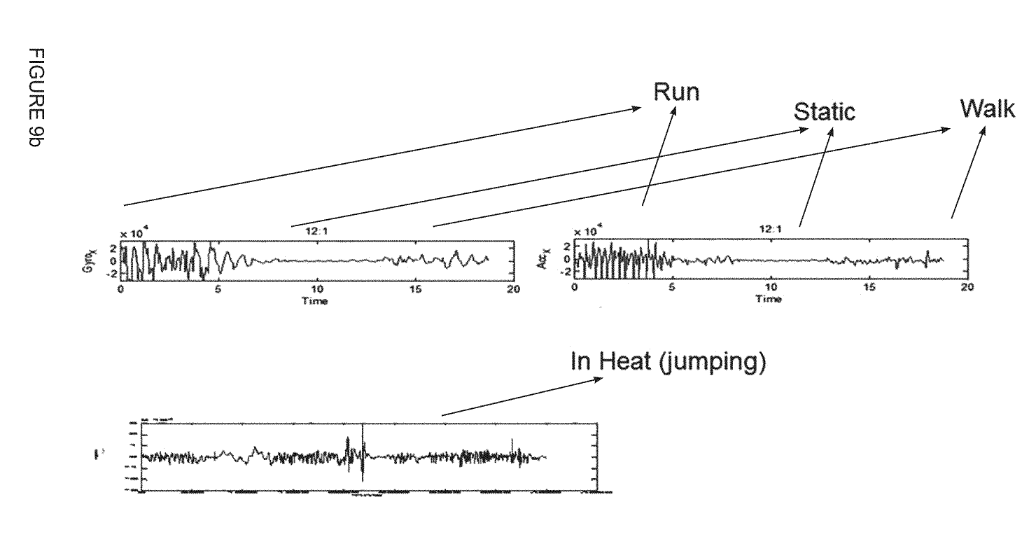

One exemplary embodiment is shown in FIGS. 9a and 9b, which illustrate graphically the plots of the sensed data that permits the determination that an animal is walking/running/static, grazing or in heat. Exemplary plots of real-time processing of sensor results, indicating behaviors of an animal, according to embodiments of the invention, are shown, graphically, in FIG. 9a, based on observing and collecting data from cows and herds during their daily routine. The plot of the 3 axis gyro results displayed on the graphs on the left side and the plot of the 3 axis accelerator results displayed on the graphs on the right side illustrate the cow's ordinary activities—running, standing, walking. In comparison, the activity displayed in FIG. 9b on the graph on the bottom shows sensor results indicating different behavior of an animal, i.e., jumping. Thus, since the symptoms of cows in heat include wrangling with each other; jumping one on the other; poking their heads into the other's backside, and allowing other cows to do these actions, it can be concluded from this jumping activity that this cow was in heat at the time these measurements were taken.

The collars that are suitable for this invention can be one of several types. One type is a satellite or cellular based mobile hub collar, suitable for both a bull and a cow, which includes a plurality of photovoltaic solar panels, for example, four panels that are 6×12 cm², each generating 4.1V; an electronics panel with a 3-axis acceleration sensor, a 3 axis gyro sensor, inertial sensors, a GPS or other geographic location device; a one- or two-way communication system transceiver, for communication within the herd over the in-herd network; a power source, such as rechargeable batteries that preferably provide at least 2 days independent operation; a processor with a non-volatile memory; a satellite or cellular modem; all mounted in a belt with a weight to hold it on the animal's neck in the correct orientation, i.e., with the electronics panel on top of the animal's spinal column in a roughly horizontal position. The collar for the bull preferably will include all these elements, although not all are required.

According to exemplary embodiments of the system, the deployment of the collars and ear tags is as follows. 5-7% of cellular or iridium mobile hub collars and 93-95% simple collars and/or ear tags. Preferably, all of the bulls will be equipped with cellular or satellite hub collars.

Operation of the system of the present invention is as follows. First, data of selected physical parameters is collected. Each collar will monitor the animal's parameters periodically, at pre-defined time intervals, for a pre-defined length of time, for example, for a few minutes each hour, and store the information collected. It is possible to change the time between periodic monitoring, or rate of sampling, when the results of the analysis meet a pre-defined criterion for a selected monitored physical condition or behavior, indicating a suspicious situation. When this pre-defined criterion has been met, a pre-defined change will be implemented. For example, if a cow shows distress, information will be collected after shorter time intervals. This change can be implemented automatically by the server or by the hub device and/or remotely by the user.

The in-herd wireless communication system periodically collects this information from the in-herd collars or ear tags on the animals via mobile hub collars on some individual animals or via a terminal disposed where the animals congregate. The mobile hub collar or terminal, in turn, will periodically transmit the data it collected and received via the in-herd communication system via a cellular or GPRS or iridium satellite or other communications network, to the Internet cloud from which it can be accessed by the user. It will be appreciated by those skilled in the art, that the periods of time when data is transmitted to the server also can be selectively controlled, either automatically or by the user, when pre-defined criteria are met.

All the simple (non-solar powered, short range transmission) collars and ear tags communicate with the mobile hub collars and/or local terminals to create the in-herd wireless mesh network that provides valuable, near-real-time insight regarding animal behavior, including herd location, walking time, grazing time, resting time, water consumption, in-heat condition, and other health events. The sensors in the collars or ear tags on the animals collect information frequently, for example, every 30 seconds, and the data is stored in the memory of the data collection device. As stated above, this information is transmitted over the in-herd wireless communication system to a mobile hub collar or local terminal, periodically during the day, for example, every four minutes, or whenever the animal passes within range of a mobile hub data collection device or a terminal. The mobile hub collar and/or local terminal transmits all the collected data periodically during the day, for example, every 4 hours, to an Iridium satellite, or other suitable communications satellite, or cellular or GPRS base station, that transmits it to the server in the Internet cloud, where it is analyzed, and the results can be accessed on the user's PC, cell phone, tablet, or other electronic device in almost real time.

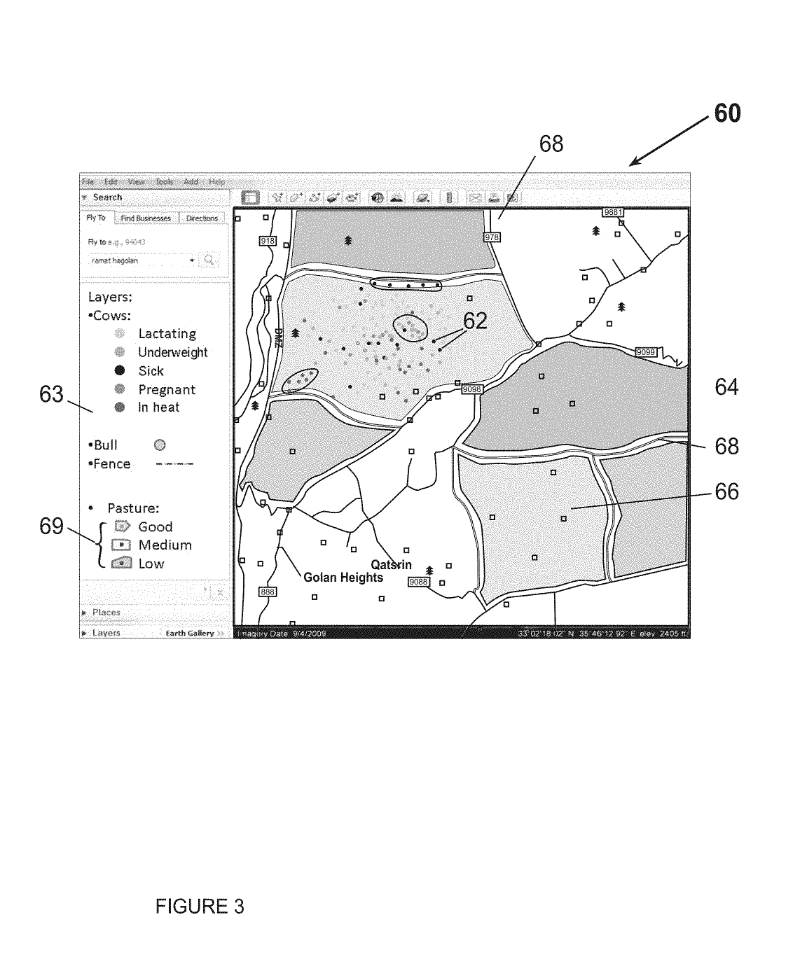

The physical parameter data is analyzed in the hub collars or terminals or on the remote server and is organized in a fashion that is user friendly, for display on one or more electronic communication devices of the user. See, for example, FIG. 3, an exemplary screen shot 60 displaying data representing the location of each animal 62 in a remote herd on the display of a personal lap-top computer. Each animal preferably is displayed as a symbol or in a color that represents a particular physical condition or behavior, for example, lactating, underweight, sick, pregnant, in heat, etc. for cows, according to a pre-defined map legend or key 63. As can be seen, in this embodiment, the location of each animal 62 is indicated by representative symbols on a computer-generated map 64, which also shows pastures 66 and fences 68. Preferably, an indication 70 is provided as to the quality of the pasture land, also as shown in legend 63.

Geographical location and tracking can be determined using GPS systems, for bulls and cows wearing the central hub devices or collars having built-in GPS equipment. The location of the cows and calves wearing the collars and ear tags without GPS can be accomplished by triangulation, by measuring the strength of the in-herd communication signals for each ear tag relative to various mobile hub collars. As with the data regarding the animal's movements, the geographical location data is analyzed and stored on the server in the Internet cloud. From there, it can be accessed by the user from any computing or communication device that has access to the Internet.

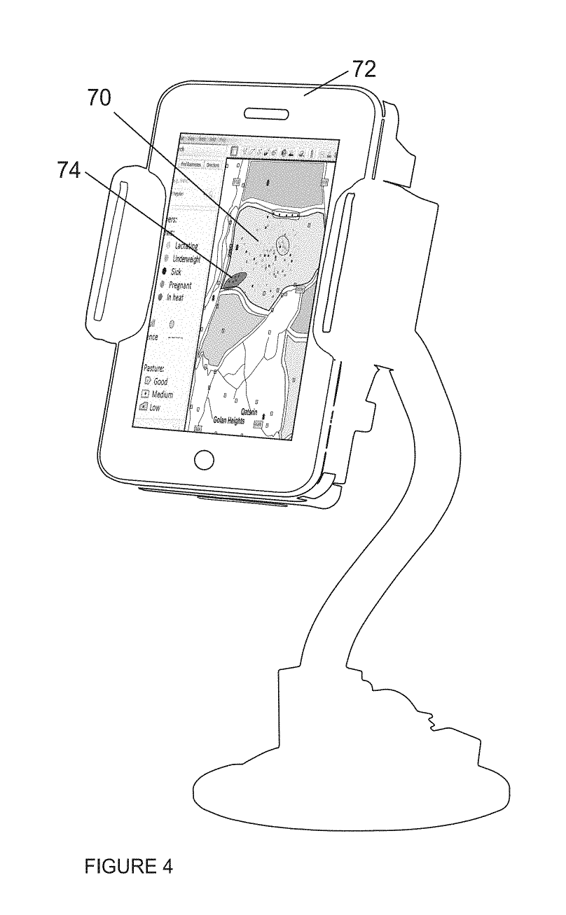

The collars and ear tags are designed to work in all weather conditions, both day and night. In addition to analyzing data regarding position and body movement of the animals, the system provides early warning alarms when illness, predatory animals, poachers or other hostile events are determined from the collected sensor data. These alarms are sent from the server in the cloud, via means for two-way communication between the remote server and the remote electronic communication device configured to provide real time information and warning alarms to the remote electronic communication device, or from a hub device, if it performs preliminary processing, and can be sent directly to the user's cell phone or other electronic device. FIG. 4 is an exemplary screen shot displaying data representing a remote herd 70 on a cellular telephone 72 including an alarm indication. In the illustrated embodiment, the display of the cellular phone shows a problematic area 74 indicated in red or otherwise highlighted, as by flashing light or expanding ripples or an audible alarm. This allows the user to take the necessary action to clarify or solve the problem. For example, the user can send a flight command to the server to operate a drone from his or her cell phone. The drone flies to the relevant location and transmits video in real time of the events occurring in the field back to the user's cell phone and/or PC. The drone's flight can be controlled automatically by the server so no manual intervention is required, although a manual override can be provided, if desired. The drone can be programmed to return, automatically, to land at the location from which it took off. The drone can use visible and/or IR imaging, e.g., using a thermal camera, to follow the herd/individual animal both during the day and at night.

It will be appreciated that, over all, the system enables ranchers to increase yields (calf delivery) up to 25% while reducing operation cost and improving pasture management.

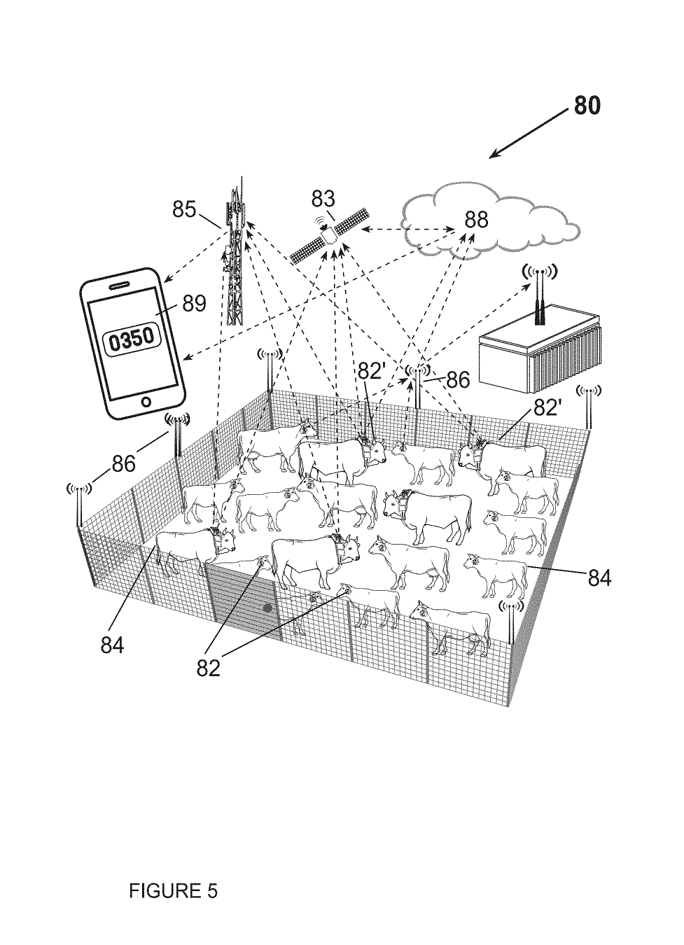

Referring now to FIG. 5, there is shown a schematic illustration of the use of the system of the present invention as a feedlot theft early warning system 80. In this embodiment, a plurality of simple data collection devices 82 are provided on most of the livestock 84. A few animals may be given mobile hub central data collection devices 82′. A plurality of terminals 86 are mounted in the fence walls holding the livestock. The data collection devices 82 transmit their collected data over a short distance and it is received and stored when the animal passes by the animals bearing the mobile hub devices and/or a terminal 86.

The mobile hub data collection devices 82′ and the terminals 86 are in two-way communication with a dedicated server 88 in the Internet, where the sensed and collected data is analyzed, substantially as described above. In case of disturbance among the animals, for example due to the entrance of unauthorized persons, the change in the livestock behavior, calculated from the sensed physical parameters, is noted and a warning sent to the user via means for two-way communication (for example, a satellite 83 or cellular or GPRS base station 85) between the remote server and a remote electronic communication device 89 of the user. The two-way communication means are configured to provide real time information and warning alarms to the remote electronic communication device.

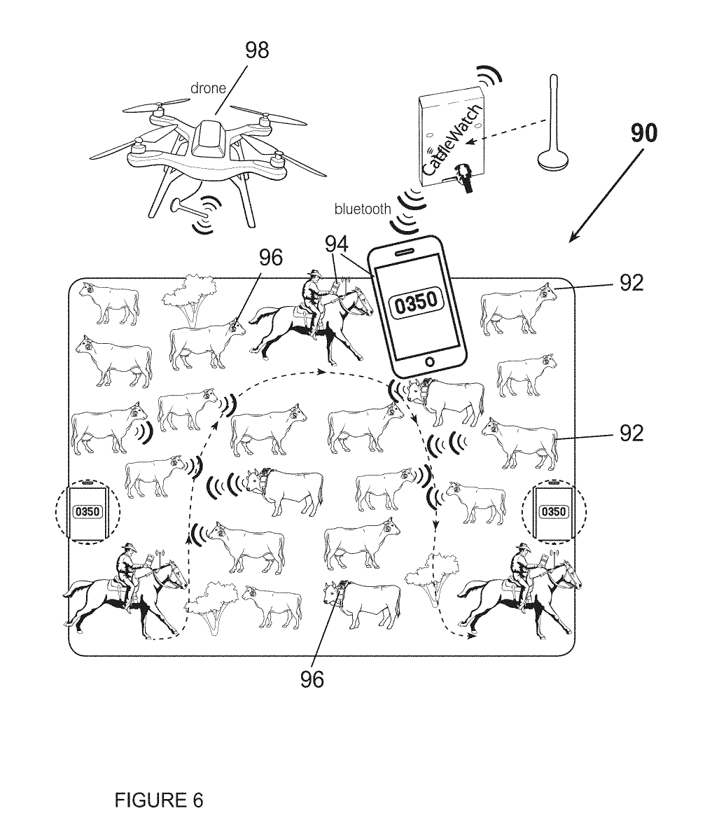

FIG. 6 is a schematic illustration of use of the system 90 for counting heads of livestock. It is sufficient for a rancher to ride in proximity to the cattle 92 in order to retrieve, in a portable or hand-held receiver 94 data from the simple data collection devices 96 on the herd. The identifying information provided in the data collection device ensures that the receiver 94 does not count the same animal more than once. Bluetooth connectivity or the like is provided between the data collection device 96 and the user's smart phone or other hand-held receiver 94. The system can be used in conjunction with a drone 98 for automated counting.

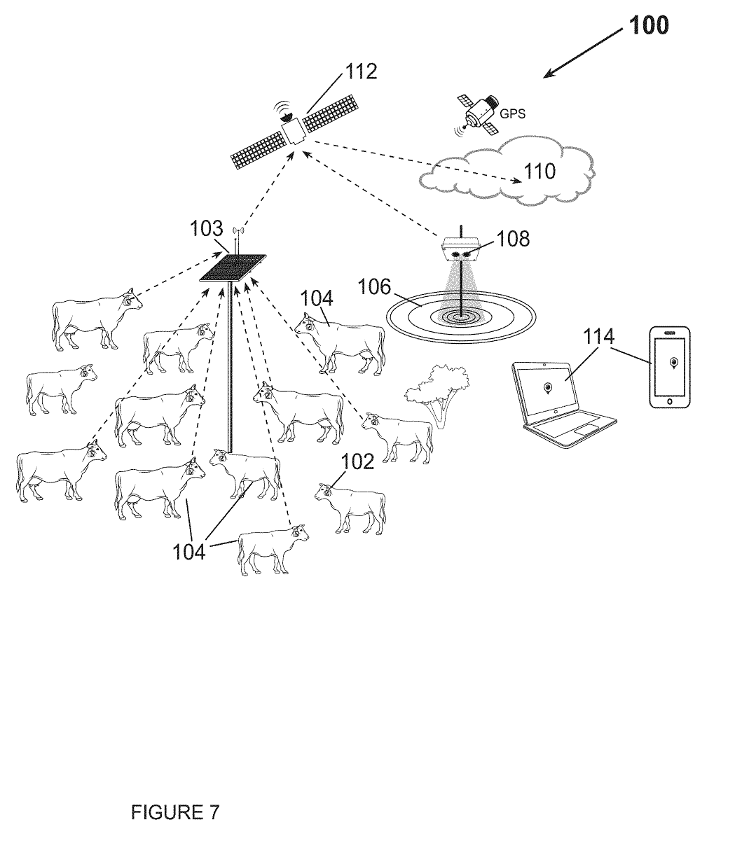

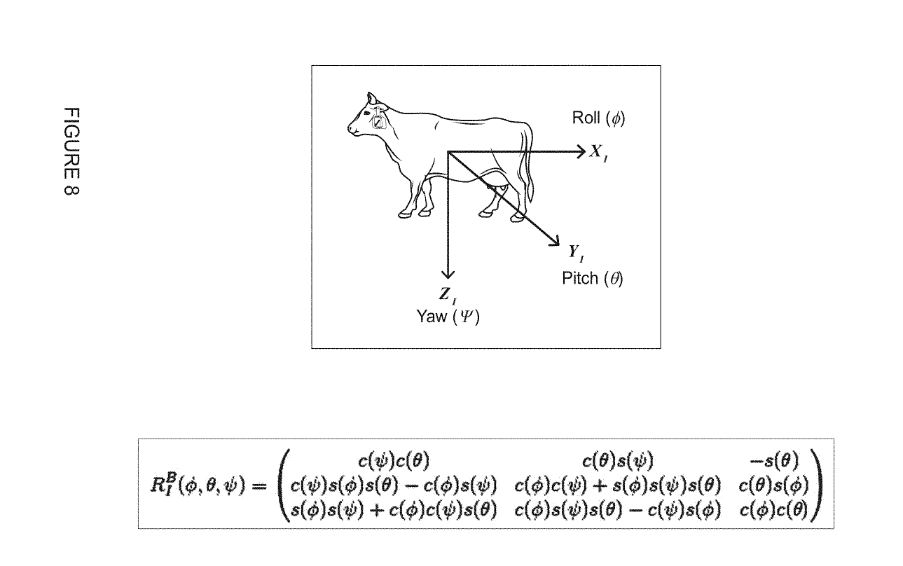

Referring now to FIG. 7, there is shown a system 100 to monitor the level of water in watering holes, rivers and streams or other drinking holes 106 in the vicinity of the livestock. This can be accomplished, for example, by analysis of head movements and attitudes of the animals 104, or example, how low the cow must bend to reach water to drink. Alternatively or in addition, this can be accomplished by using a water level meter 108. In this embodiment, a water sensor 108 is provided to improve the measurement of the level of water, in addition to the calculations provided by the sensors of the movements of the livestock body or head during or before drinking. These movements are sensed by the sensors in the data collection devices 102 on the animals (such as those described above), which are transmitted via a central terminal 103 to a server 110, either directly or via a communication network 112, here shown as a satellite network. Here, too, the data is analyzed and the results are accessible in real time by a user via his or her computing device 114, e.g., laptop or smart phone.

In some embodiments of the current invention, the sensed data can be analyzed to determine whether an animal in the herd is in heat (estrus), conception date of at least one animal of the herd, expected calving date of at least one animal of the herd, or breeding activities, i.e., interaction between a male animal and a female in the herd.

If desired, the sum of the daily activity of each animal can be stored during the course of the animal's lifetime. This data can be further analyzed off-line, in the server or by the user or in any other fashion, and can be used, inter alia, to provide statistics of the herd over time. For example, in addition to health events of an individual cow, the data can indicate health events of the herd (epidemics, etc.) Thus, health events of individuals can be determined, for example, if there is a reduction of both, daily grazing time and distance for an individual, as compared to a herd average of daily grazing time and distance on previous days, which remained substantially constant, unless other behavior (like coming calving) is expected. Health events in the herds (epidemics) will be indicated when, from day to day, more and more animals show behavior indicating illness, while the rest of the monitored herd behavior of daily grazing time and distance traveling and walking idle time remains similar from day to day.

Estrus of a cow can be detected by data indicating that the cow moves more and eats less than during previous days. Thus, when an individual cow travels a longer distance and grazes for a shorter time than her average over the preceding days, it can be concluded that she is in heat. Similarly, the ratio of daily walking time to daily grazing time will increase.

Since cows are in a cycle of heat every 19 to 22 days and the duration of pregnancy for a cow is almost constant (280 days), if the cow does not repeat the behavior of being in heat at an interval of about 19 to 23 days, and the behavior of the rest of the herd has not deteriorated significantly, it can be concluded that the cow successfully conceived during the previous estrus cycle and, consequently, the expected date of calving is 280 days from the heat detection date. On the other hand, identifying a short period (about 15 days or less) between two events of heat is an indication of a problem in the ovaries (e.g., cysts).

The present invention also permits the monitoring of breeding bulls' activities and of the interactions between bulls and cows. In the reproduction season, several breeding bulls may be introduced to the herd of cows. It is very important to know which of them are active and mate with the cows. For this purpose, a cow proximity identifier can be disposed in the bulls' collars to provide an indication when a cow is near the bull (e.g., a distance shorter than about 40 cm). Identifying a relatively long time (longer than about 5 minutes) of proximity to a number of cows in heat during the daylight hours in the geographical area where most of the herd is grazing (not resting), or in a location far from the herd's resting area, will be an indication of good activity of the specific individual breeding bull. This can be cross correlated to detection of cows' reproduction activity and the following expected calving date.

While the invention has been described with respect to a limited number of embodiments, it will be appreciated that many variations, modifications and other applications of the invention may be made. It will further be appreciated that the invention is not limited to what has been described hereinabove merely by way of example. Rather, the invention is limited solely by the claims which follow.

1. A system for monitoring livestock, the system comprising:
a remote server storing data, the remote server including a processing unit for processing stored data and a non-volatile memory;
at least one simple data collection device for mounting on an animal to be monitored, the data collection device including a sensor sensing physical parameters of the animal on which it is mounted, a simple data collection device processor with a non-volatile memory, and a transmitter for transmitting data collected by the sensor;
at least one central data collection device including a mobile hub for mounting on an animal to be monitored, the central data collection device including:
a central data collection device processor with a non-volatile memory;
a receiver for receiving data transmitted by the simple data collection devices;
a transceiver for communication with the remote server over a communication system;
an energy source;
the remote server being configured to analyze collected physical parameters and determine therefrom a pre-defined physical condition or behavior of the animal; and
means for two-way communication between the remote server and a remote electronic communication device and configured to provide the analyzed data in real time and warning alarms to the remote electronic communication device.

2. The system according to claim 1, wherein:
at least one simple data collection device includes a plurality of simple data collection devices; and
at least one central data collection device includes a plurality of central data collection devices.

3. The system according to claim 1, further comprising a drone operated by said remote server.

4. The system according to claim 1, further comprising a warning mechanism arranged to send a warning alarm to the remote electronic communication device when a pre-defined animal behavior or physical condition is determined by the remote server or the central data processing device processor.

5. The system according to claim 1, further comprising a communication network for two-way communication between central data collection devices and the remote server, and between the server and a remote electronic communication device, wherein the communication system is selected from the group including Iridium satellites, communication satellites, cellular network, GPRS network.

6. The system according to claim 1, wherein:
said remote electronic communication device includes a display for displaying results from the server of analysis of the collected data; and
said displayed results include symbols representing pre-defined physical conditions or behaviors of the animals to be monitored and said displayed results include a legend.

7. The system according to claim 1, wherein each data collection unit includes a 3 axis accelerator and a 3 axis gyro providing data to the data collection unit processor.

8. The system according to claim 7, wherein the processing unit of the remote server is configured to calculate a data collection unit attitude, which reflects a neck attitude, from sensed data from the 3 axis accelerator and the 3 axis gyro by the following Direction Cosine Matrix (DCM):
$${R}_{I}^{B} ( φ , θ , ψ ) = ( \begin{matrix}c ( ψ ) c ( θ ) & c ( θ ) s ( ψ ) & - s ( θ ) \\ c ( ψ ) s ( φ ) s ( θ ) - c ( φ ) s ( ψ ) & c ( φ ) c ( ψ ) + s ( φ ) s ( ψ ) s ( θ ) & c ( θ ) s ( φ ) \\ s ( φ ) s ( ψ ) + c ( φ ) c ( ψ ) s ( θ ) & c ( φ ) s ( ψ ) s ( θ ) - c ( ψ ) s ( φ ) & c ( φ ) c ( θ )\end{matrix} )$$
where ϕ represents the roll, in the X axis, θ represents the pitch, in the Y axis, and ψ represents the yaw, in the Z axis, of the data collection device.

9. The system according either claim 7, wherein the processor is further configured to calculate a water level in a water source from which the animal to be monitored is drinking.

10. The system according to claim 7, wherein the central data collection device includes a solar-powered rechargeable battery.

11. The system according to claim 7, wherein a central data collection device mounted on a bull further comprises a cow proximity identifier to provide an indication when a cow is near the bull.

12. The system according to claim 7, further comprising means for processing collected data of selected physical parameters, determining whether results of processing meet a pre-defined threshold and, if so, changing the pre-defined time interval of collecting data.

13. The system according to claim 7, wherein the simple data collection devices are mobile data collection devices.

14. A method for monitoring animals to be monitored, the method comprising:
collecting, at pre-defined time intervals for pre-defined periods of time, data of physical parameters of an animal to be monitored sensed by a sensor in a simple data collection device mounted on the animal to be monitored;
storing the collected data in the simple data collection device;
transmitting stored collected data by the simple data collection device to a mobile hub device at pre-defined time intervals;
receiving, in the mobile hub device, the data transmitted by the simple data collection device,
transmitting, by the mobile hub device, the received data to a remote server;
analyzing the transmitted data to determine physical condition and behavior in the remote server and storing the analyzed data; and
permitting access to said stored data in the remote server by at least one remote electronic communication device, said remote server transmitting real time information and warning alarms to the remote electronic communication device.

15. The method according to claim 14, wherein a data collection device attitude, which reflects a neck attitude, is calculated from sensed data from a 3 axis accelerator and a 3 axis gyro in the data collection device by the following Direction Cosine Matrix (DCM):
$${R}_{I}^{B} ( φ , θ , ψ ) = ( \begin{matrix}c ( ψ ) c ( θ ) & c ( θ ) s ( ψ ) & - s ( θ ) \\ c ( ψ ) s ( φ ) s ( θ ) - c ( φ ) s ( ψ ) & c ( φ ) c ( ψ ) + s ( φ ) s ( ψ ) s ( θ ) & c ( θ ) s ( φ ) \\ s ( φ ) s ( ψ ) + c ( φ ) c ( ψ ) s ( θ ) & c ( φ ) s ( ψ ) s ( θ ) - c ( ψ ) s ( φ ) & c ( φ ) c ( θ )\end{matrix} )$$
where ϕ represents the roll, in the X axis, θ represents the pitch, in the Y axis, and ψ represents the yaw, in the Z axis, of the data collection device.

16. The method according to claim 14 or claim 15, further comprising:
sending a drone to fly over the animal to be monitored; and
receiving images of the animal from the drone in real time.

17. The method according to claim 14, further comprising:
mounting a central data collection device on a bull including a cow proximity identifier;
receiving an indication when a cow is near the bull;
identifying a relatively long time (longer than about 5 minutes) of proximity to a number of cows in heat during pre-defined times in a pre-defined geographical area as an indication of good activity of the specific bull.

18. The method according to claim 17, further comprising cross correlating activity of the bull to detection of cows' reproduction activity and expected calving dates.

19. The method according to claim 14, further comprising:
collecting data of selected physical parameters in data collection device periodically, at pre-defined time intervals, for a pre-defined length of time;
analysing the collected information;
examining results of analysis to determine if meet pre-defined criteria regarding a selected monitored physical condition or behavior; and
if the pre-defined criteria are met, changing the pre-defined time intervals.

20. (canceled)

21. A system for monitoring livestock, the system comprising:
a remote server storing data in the Internet cloud, the remote server including a processing unit;
at least one simple data collection device for mounting on an animal to be monitored, the data collection device including at least two sensors sensing physical parameters of the animal on which it is mounted and a transmitter for transmitting data collected by the sensors;
at least one central data collection device selected from the group including: a mobile hub for mounting on an animal to be monitored or a local terminal, the central data collection device including:
a central data collection device processor with a non-volatile memory;
a receiver for receiving data transmitted by the simple data collection devices;
a transceiver for communication with the remote server over a communication system;
an energy source; and
the remote server being configured to analyze collected physical parameters and determine therefrom a pre-defined physical condition or behavior of the animal; and
means for two-way communication between the remote server and a remote electronic communication device and configured to provide the analyzed data in real time and warning alarms to the remote electronic communication device.
